(19) United States
(12) Patent Application Publication
(10) Pub. No.: US 2009/0048198 A1
(43) Pub. Date: Feb. 19, 2009

(54) COMPOSITIONS AND METHODS FOR NUCLEIC ACID DELIVERY SYSTEMS

(75) Inventors: Rumiana Koynova-Tenchova, Columbus, OH (US); Li Wang, Fremont Lane, CA (US); Robert C. MacDonald, El Cerrito, CA (US)

Correspondence Address: Casimir Jones, S.C., 440 Science Drive, Suite 203, Madison, WI, 53711, US

(73) Assignee: Northwestern University, Evanston, IL (US)

(21) Appl. No.: 12/040,977

(22) Filed: Mar. 3, 2008

Related U.S. Application Data
(60) Provisional application No. 60/904,607, filed on Mar. 2, 2007.

(51) Int. Cl. A61K 31/7088; C12N 15/64; A61K 47/12; A61P 43/00; C12N 15/88

(52) U.S. Cl. 514/44; 435/320.1; 514/784; 435/458

(57) ABSTRACT

The present invention relates to compositions and methods for use in delivering nucleic acids and other agents into cells and tissues. In particular, the present invention provides lipid mixtures at the gel-liquid crystalline phase transition providing superior lipofection activity for transferring materials into cells and tissues.

The present application claims priority to U.S. Provisional Application 60/904,607, filed Mar. 2, 2007, which is herein incorporated by reference in its entirety.

The invention was made with government support under grants GM52329, GM57305 and U54CA119341 warded by the National Institutes of Health. The government has certain rights in this invention.

### FIELD OF THE INVENTION

The present invention relates to compositions and methods for use in delivering nucleic acids and other agents into cells and tissues. In particular, the present invention provides lipid mixtures at the gel-liquid crystalline phase transition providing superior lipofection activity for transferring materials into cells and tissues.

### BACKGROUND OF THE INVENTION

To date, the primary approach to improving the transfection properties of cationic lipids has been the synthesis of new kinds of cationic amphipaths or the inclusion of non-cationic helper lipids. While such approaches have met with some success, improved transfection reagents that provide efficient transfection (e.g., efficient nucleic acid uptake, low toxicity) are needed.

### SUMMARY OF THE INVENTION

The present invention relates to compositions and methods for use in delivering nucleic acids and other agents into cells and tissues. In particular, the present invention provides lipid mixtures at the gel-liquid crystalline phase transition providing superior lipofection activity for transferring materials into cells and tissues.

Combination of two cationic phospholipid compounds has been found able to generate a lipid carrier with greater ability to efficiently deliver therapeutic agents such as nucleic acids (e.g, DNA, RNA) than is achievable by the separate molecules. In part, the increased delivery activity is attributed to phase changes that result from exposure to cellular conditions. In developing embodiments of the present invention, it was found that, in addition to the cationic O-ethylphosphatidylcholines, quite a large proportion of common, commercially available cationic lipid delivery agents, such as dimethylammonium bromides and trimethylammonium propanes, exhibit such “mixture synergism”. In an effort to rationalize this widespread synergy, phase behavior of the cationic lipid mixtures and constructed their binary phase diagrams was examined. The compositions at which the mixtures exhibit maximum delivery activity were found to clearly coincide with those regions of the phase diagram where the mixture resides within the solid-liquid crystalline two-phase region at physiological temperature.

Thus, the transfection efficacy of formulations exhibiting solid-liquid crystalline phase coexistence is more than 5× higher relative to formulations in the gel (solid) phase, and more than twice higher relative to liquid crystalline formulations. It is contemplated that packing defects at the boundaries of the coexisting solid and liquid crystalline domains is responsible for enhanced fusogenicity of the mixtures.

As such, the present invention provides compositions and method for delivery of agents by manipulating the composition of the lipid carriers so as to adjust their phase transition to take place at physiological temperature or other desired temperatures, thereby providing a valuable tool to enhance their delivery efficacy.

In one embodiment, the present invention provides a composition for use as a nucleic acid delivery system comprising a mixture of lipids wherein the mixture resides within the solid-liquid crystalline two-phase region of a binary phase diagram at physiological temperature.

In some embodiments, the mixture of lipids comprises one or more of EDMPC and EDPPC, diC14DAB and diC18DAB, EDOPC and diC14DAB, EDOPC and diC18DAB, and EDOPC and DMTAP.

In certain embodiments, the mixture of lipids comprises EDMPC and EDPPC, wherein 70-80 mol %, or 65-85 mol %, of the mixture of lipids is EDPPC (e.g., 65, 66, 67, 68, 69, 70, 71, 72, 73, 74, 75, 76, 77, 78, 79, or 80 mol %). In some embodiments, the mixture of lipids comprises EDMPC and EDPPC, and about 70 mol % of the mixture of lipids is EDPPC. In particular embodiments, the mixture of lipids comprises EDMPC and EDPPC, wherein 20-30 mol %, or 15-35 mol %, of the mixture of lipids is EDMPC (e.g., 15, 16, 17, 18, 19, 20, 21, 22, 23, 24, 25, 26, 27, 28, 29, or 30 mol %).

In some embodiments, the mixture of lipids comprises diC14DAB and diC18DAB, wherein 50-90 mol %, or 40-95 mol %, of the mixture of lipids is diC18DAB (e.g., 40 . . . 50 . . . 60 . . . 70, 71, 72, 73, 74, 75, 76, 77, 78, 79, 80, . . . 95 mol %). In some embodiments, the mixture of lipids comprises diC14DAB and diC18DAB, and about 75 mol % of the mixture of lipids is diC18DAB. In further embodiments, the mixture of lipids comprises diC14DAB and diC18DAB, wherein 10-50 mol %, of the mixture of lipids is diC14DAB (e.g., 10 . . . 20 . . . 30 . . . 40 . . . 50 mol %).

In particular embodiments, the mixture of lipids comprises EDOPC and diC14DAB, wherein 60-80 mol %, or 50-90 mol %, of the mixture of lipids is diC14DAB (e.g., 60 . . . 70 . . . 75, 76, 77, 78, 79, 80, 81, 82, 83, 84, 85 . . . 90 mol %). In other embodiments, the mixture of lipids comprises EDOPC and diC14DAB, and about 80 mol % of the mixture of lipids is diC14DAB. In some embodiments, the mixture of lipids comprises EDOPC and diC14DAB, wherein 20-40 mol % of the mixture of lipids is EDOPC (e.g., 20, 21, 22, 23, 24, 25, 26, 27, 28, 29, 30 . . . 40 mol %).

In particular embodiments, the mixture of lipids comprises EDOPC and diC18DAB, wherein 10-90 mol %, or 5-95 mol %, of the mixture of lipids is diC18DAB (e.g., 5, 6, 7, 8, 9, 10, 11, 12, 13, 14, 15 . . . 25 . . . 50 . . . 75 . . . 90 . . . 95 mol %). In some embodiments, the mixture of lipids comprises EDOPC and diC18DAB, and 10-90 mol % of the mixture of lipids is EDOPC (e.g., 10 . . . 50 . . . 85, 86, 87, 88, 89, or 90 mol %).

In additional embodiments, the mixture of lipids comprises EDOPC and DMTAP, wherein 20-60 mol %, or 10-70 mol %, of the mixture of lipids is DMTAP (e.g., 10 . . . 20, 21, 22, 23, 24, 25, 26, 27, 28, 29, 30 . . . 50 . . . 60 . . . 70 mol %). In some embodiments, the mixture of lipids comprises EDOPC and DMTAP, wherein about 25 mol % of the mixture of lipids is DMTAP. In other embodiments, the mixture of lipids comprises EDOPC and DMTAP, wherein 40-80 mol % of the mixture of lipids is EDOPC (e.g., 40 . . . 60 . . . or 80% mol).

In particular embodiments, the present invention provides compositions comprising EDMPC and EDPPC, wherein 70-80 mol % of the composition is EDPPC (e.g., about 70 mol % EDPPC). In certain embodiments, the compositions comprise diC14DAB and diC18DAB, wherein 50-90 mol % of the composition is diC18DAB (e.g., about 75 mol % diC18DAB). In further embodiments, the compositions comprise EDOPC and diC14DAB, wherein 60-80 mol % of the composition is diC14DAB (e.g., about 80 mol % diC14DAB). In further embodiments, the compositions comprise EDOPC and diC18DAB, wherein 10-90 mol % of the composition is diC18DAB (e.g., about 10 mol % diC18DAB). In some embodiments, the compositions comprise EDOPC and DMTAP, wherein 20-60 mol % of the composition is DMTAP (e.g., about 25 mol % DMTAP).

In particular embodiments, the compositions of the present invention further comprise a nucleic acid molecule, an expression vector, a drug, or combinations thereof.

In one embodiment, the present invention provides a method of transfecting a cell comprising, exposing a cell to the composition for use as a nucleic acid delivery system comprising a mixture of lipids wherein the mixture resides within the solid-liquid crystalline two-phase region of a binary phase diagram at physiological temperature, wherein said composition further comprises a nucleic acid molecule. The present invention is not limited by the type of cell or tissue transfected as all types of cells and tissues are contemplated. In certain embodiments, cells that are known to be difficult to transfect, such as human endothelial cells, are successfully transfected (e.g., at high percentages such as those reported in the Examples). In certain embodiments, the types of cells transfected include, but are not limited to, keratinizing epithelial cells, wet stratified barrier epithelial cells, exocrine secretory epithelial cells, hormone secreting cells, metabolism and storage cells, barrier function cells (Lung, Gut, Exocrine Glands and Urogenital Tract), kidney, epithelial cells lining closed internal body cavities, ciliated cells with propulsive function, extracellular matrix secretion cells, contractile cells, blood and immune system cells, sensory transducer cells, autonomic neuron cells, sense organ and peripheral neuron supporting cells, central nervous system neurons and glial cells, lens cells, pigment cells, germ cells, nurse cells, and interstitial cells. In some embodiments, the transfected cell is in vitro, while in other embodiments the transfected cell is in vivo.

In one embodiment, the present invention provides a method for selecting a lipid mixture that provides high transfection efficiency in a delivery system, comprising selecting a first lipid and a second lipid and combining a ratio of said first lipid and second lipid in a transfection reagent, said ratio optimized to cause said first and second lipids to exist within a solid-liquid crystalline two-phase region of a binary phase diagram at physiological temperature.

In one embodiment, the present invention provides a composition comprising a delivery system selected by the method of selecting a lipid mixture that provides high transfection efficiency in a delivery system, comprising selecting a first lipid and a second lipid and combining a ratio of said first lipid and second lipid in a transfection reagent, said ratio optimized to cause said first and second lipids to exist within a solid-liquid crystalline two-phase region of a binary phase diagram at physiological temperature.

### DEFINITIONS

To facilitate an understanding of the present invention, a number of terms and phrases are defined below:

As used herein the term “lipoid” refers to any natural or synthetic lipid molecules, including non-natural compounds that are similar to lipids in structure and properties (e.g., they are amphipaths).

As used herein the term “transfection efficiency” refers to, for example, the percentage of target cells, within a population of target cells, which contain an introduced exogenous nucleic acid molecule. Transfection efficiency can be determined by transfecting a nucleic acid molecule encoding a reporter gene into a population of target cells and determining the percentage of cells having reporter activity. The term “transfection efficiency” also refers to the amount of gene product detected following transfection of the nucleic acid into the cell. This is determined, for example, by testing an entire cell population for the amount of gene product produced after a given incubation period. Thus, the term “transfection efficiency” involves assaying for the relative expression of the gene product encoded by the introduced nucleic acid.

As used herein, the term “liposome” refers to a vesicle bounded by a lipid bilayer. A “cationic liposome” has a net positive charge.

As used herein, the term “short chain fatty acid” refers to a fatty acid chain having 7 or less carbons.

As used herein, the term “medium chain fatty acid” refers to a fatty acid chain having between 8 and 15 carbons (e.g., laurate, myristate, etc.).

As used herein, the term “long chain fatty acid” is a fatty acid chain having 16 or more carbons (e.g., palmitate, stearate, oleate, etc.).

As used herein, the term “nucleic acid molecule” refers to any nucleic acid containing molecule including, but not limited to DNA or RNA. The term encompasses sequences that include any of the known base analogs of DNA and RNA including, but not limited to, 4-acetylcytosine, 8-hydroxy-N6-methyladenosine, aziridinylcytosine, pseudoisocytosine, 5-(carboxyhydroxylmethyl) uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxyaminomethyl-2-thiouracil, beta-D-mannosylqueosine, 5′-methoxycarbonylmethyluracil, 5-methoxyuracil, 2-methylthio-N6-isopentenyladenine, uracil-5-oxyacetic acid methylester, uracil-5-oxyacetic acid, oxybutoxosine, pseudouracil, queosine, 2-thiocytosine, 5-methyl-2-thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine.

The term “gene” refers to a nucleic acid (e.g., DNA) sequence that comprises coding sequences necessary for the production of a polypeptide or precursor. The polypeptide can be encoded by a full length coding sequence or by any portion of the coding sequence so long as the desired activity or functional properties (e.g., enzymatic activity, ligand binding, signal transduction, etc.) of the full-length or fragment are retained. The term also encompasses the coding region of a structural gene and the including sequences located adjacent to the coding region on both the 5′ and 3′ ends for a distance of about 1 kb or more on either end such that the gene corresponds to the length of the full-length mRNA. The sequences that are located 5′ of the coding region and which are present on the mRNA are referred to as 5′ non-translated sequences. The sequences that are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

As used herein, the term “gene expression” refers to the process of converting genetic information encoded in a gene into RNA (e.g., mRNA, rRNA, tRNA, or snRNA) through “transcription” of the gene (i.e., via the enzymatic action of an RNA polymerase), and for protein encoding genes, into protein through “translation” of mRNA. Gene expression can be regulated at many stages in the process. “Up-regulation” or “activation” refers to regulation that increases the production of gene expression products (i.e., RNA or protein), while “down-regulation” or “repression” refers to regulation that decrease production. Molecules (e.g., transcription factors) that are involved in up-regulation or down-regulation are often called “activators” and “repressors,” respectively.

The term “transgene” as used herein refers to a foreign gene that is placed into an organism by, for example, introducing the foreign gene into newly fertilized eggs or early embryos. The term “foreign gene” refers to any nucleic acid (e.g., gene sequence) that is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. The term “vehicle” is sometimes used interchangeably with “vector.” Vectors are often derived from plasmids, bacteriophages, or plant or animal viruses.

The term “expression vector” as used herein refers to a recombinant DNA molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed (e.g., the amount of 28S rRNA, an abundant RNA transcript present at essentially the same amount in all tissues, present in each sample can be used as a means of normalizing or standardizing the mRNA-specific signal observed on Northern blots). The amount of mRNA present in the band corresponding in size to the correctly spliced transgene RNA is quantified; other minor species of RNA which hybridize to the transgene probe are not considered in the quantification of the expression of the transgenic mRNA.

The term “transfection” as used herein refers to the introduction of foreign DNA into eukaryotic cells.

The term “stable transfection” or “stably transfected” refers to the introduction and integration of foreign DNA into the genome of the transfected cell. The term “stable transfectant” refers to a cell which has stably integrated foreign DNA into the genomic DNA.

The term “transient transfection” or “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectant” refers to cells which have taken up foreign DNA but have failed to integrate this DNA.

As used herein, the term “cell culture” refers to any in vitro culture of cells. Included within this term are continuous cell lines (e.g., with an immortal phenotype), primary cell cultures, finite cell lines (e.g., non-transformed cells), and any other cell population maintained in vitro.

As used, the term “eukaryote” refers to organisms distinguishable from “prokaryotes.” It is intended that the term encompass all organisms with cells that exhibit the usual characteristics of eukaryotes, such as the presence of a true nucleus bounded by a nuclear membrane, within which lie the chromosomes, the presence of membrane-bound organelles, and other characteristics commonly observed in eukaryotic organisms. Thus, the term includes, but is not limited to such organisms as fungi, protozoa, and animals (e.g., humans).

As used herein, the term “in vitro” refers to an artificial environment and to processes or reactions that occur within an artificial environment. In vitro environments can consist of, but are not limited to, test tubes and cell culture. The term “in vivo” refers to the natural environment (e.g., an animal or a cell) and to processes or reaction that occur within a natural environment.

The term “test compound” refers to any chemical entity, pharmaceutical, drug, and the like that can be used to treat or prevent a disease, illness, sickness, or disorder of bodily function. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense and includes environmental and biological samples. Environmental samples include material from the environment such as soil and water. Biological samples may be animal, including, human, fluid (e.g., blood, plasma and serum), solid (e.g., stool), tissue, liquid foods (e.g., milk), and solid foods (e.g., vegetables).

As used herein, the term “drug” refers to pharmacologically active molecules that are used to diagnose, treat, or prevent diseases or pathological conditions in a physiological system (e.g., a subject, or in vivo, in vitro, or ex vivo cells, tissues, and organs). Drugs act by altering the physiology of a living organism, tissue, cell, or in vitro system to which the drug has been administered.

### DESCRIPTION OF THE FIGURES

FIG. 1 (A) Heating thermograms of dispersions of EDMPC/EDPPC mixtures at different ratios; heating rate 30° C./h; the thermograms of the mixtures with maximum transfection activity are shown in red; (B) left Y-axis, line and symbol graph: phase diagram, constructed from calorimetric data [1,7]: squares—transition onset; circles—transition end; these were used to draw the solidus and liquidus lines, respectively, outlining the phase regions in the phase diagram: below the solidus the mixtures are in the solid phase (S); above the liquidus—in the liquid crystalline (L) phase; between the two lines there is a region of solid-liquid phase coexistence (S+L); right Y-axis, column graph: transfection efficacy TE of each composition compared to the average efficacy <TE> of all examined compositions: [(TE−<TE>)/<TE>]×100; shaded region indicate compositions, which reside within the solid-liquid crystalline two-phase region at 37° C.; (C) Transfection of HUAECs by EDMPC/EDPPC mixtures; the average efficacy <TE> is indicated with the red line.

FIG. 2 (A) Heating thermograms of dispersions of diC14DA/diC18DAB mixtures at different ratios; heating rate 30° C./h; (B) left T-axis, line and symbol graph: phase diagram, constructed from the calorimetric data: squares—transition onset; circles—transition end; right Y-axis, column graph: transfection efficacy TE of each composition compared to the average efficacy <TE> of all studied compositions: [(TE−<TE>)/<TE>]×100; shaded region indicate compositions, which reside within the solid-liquid crystalline two-phase region at 37° C.; (D) Transfection of HUAECs by diC14DA/diC18DAB mixtures; (C) SAXD patterns of diC14DAB, diC14DAB/diC18DAB 26:74 (mol/mol), and diC18DAB samples, recorded upon heating; the bolded pattern is recorded at 37° C.

FIG. 3 (A) Heating thermograms of dispersions of EDOPC/diC14DAB mixtures at different ratios; heating rate 30° C./h; (B) left T-axis, line and symbol graph: phase diagram, constructed from the calorimetric data: squares—transition onset; circles—transition end; the left part of the diagram at dominating EDOPC content is only tentatively drafted, because the EDOPC transition at subzero temperature was outside the temperature range accessible by high-sensitivity DSC; right Y-axis, column graph: transfection efficacy TE of each composition compared to the average efficacy <TE> of all studied compositions: [(TE−<TE>)/<TE>]×100; shaded region indicate compositions, which reside within the solid-liquid crystalline two-phase region at 37° C.; (D) Transfection of HUAECs by EDOPC/diC14DAB mixtures; (C) SAXD patterns of EDOPC/diC14DAB 20:80 (mol/mol), recorded upon heating; the bolded pattern is recorded at 37° C.

FIG. 4 (A) Heating thermograms of dispersions of EDOPC/diC18DAB mixtures at different ratios; heating rate 30° C./h; (B) left T-axis, line and symbol graph: phase diagram, constructed from the calorimetric data: squares—transition onset; circles—transition end; right Y-axis, column graph: transfection efficacy TE of each composition compared to the average efficacy <TE> of all studied compositions: [(TE−<TE>)/<TE>]×100; shaded region indicate compositions, which reside within the solid-liquid crystalline two-phase region at 37° C.; (D) Transfection of HUAECs by EDOPC/diC18DAB mixtures; (C) SAXD patterns of EDOPC/diC18DAB 92:8 and 15:85 (mol/mol) dispersions, recorded upon heating; the bolded pattern is recorded at 37° C.

FIG. 5 (A) Heating thermograms of dispersions of EDOPC/DMTAP mixtures at different ratios; heating rate 30° C./h; (B) left T-axis, line and symbol graph: phase diagram, constructed from the calorimetric data: squares—transition onset; circles—transition end; right Y-axis, column graph: transfection efficacy TE of each composition compared to the average efficacy <TE> of all studied compositions: [(TE−<TE>)/<TE>]×100; shaded region indicate compositions, which reside within the solid-liquid crystalline two-phase region at 37° C.; (D) Transfection of HUAECs by EDOPC/DMTAP mixtures; (C) SAXD patterns of EDOPC/DMTAP 75:25, 33:67, and 15:85 (mol/mol) dispersions, recorded upon heating; the bolded pattern is recorded at 37° C.

FIG. 6 Transfection efficacy (TE) of all studied samples of the five mixtures, compared to the average efficacy for each given mixture as: [(TE−<TE>)/<TE>]×100, plotted against the difference of the liquidus temperature for the given lipid composition and 37° C. [T(liquidus)−37° C.] (there are few cases in which given composition was falling outside the transition region, being in the solid phase at 37° C.—such as, e.g., EDMPC/EDPPC at EDPPC ≧90 mol %, etc.; for these compositions, the X-axis value was calculated as [37° C.−T(solidus)]).

### DETAILED DESCRIPTION OF THE INVENTION

Certain illustrative embodiments of the invention are described below. The present invention is not limited to these embodiments. A publication containing certain details related to this invention are provided in Koynova et al., J Phys Chem B. 2007 Jul. 12; 111(27):7786-95, which is herein incorporated by reference.

Cationic lipids are currently considered the most promising non-viral gene carriers. Major difficulty for their clinical application is the unsatisfactory efficiency. Thus far, the primary approaches to improving transfection properties of cationic lipids has been the synthesis of new kinds of cationic amphiphiles or the inclusion of non-cationic helper lipids (e.g., DOPE, cholesterol). Although both approaches have produced considerable improvement in the transfection properties of cationic lipid carriers, their transfection activity is still rather low relative to that of viral vectors, and they are currently not efficient enough to be clinically useful.

An effective alternative strategy for improving lipofection efficiency has recently emerged: the combination of two cationic lipid derivatives can synergistically enhance transfection [L. Wang, R. C. MacDonald, New Strategy for Transfection: Mixtures of Medium-Chain and Long-Chain Cationic Lipids Synergistically Enhance Transfection, Gene Ther. 11 (2004) 1358-1362]. This intriguing synergy was first observed with cationic phospholipids having the same headgroup but different hydrocarbon chains: it was noticed that the optimal combination of the long chain/medium chain lipoids, dioleoyl- and dilauroyl-ethylphosphatidylcholines (EDOPC and EDLPC, respectively), delivered DNA into human umbilical artery endothelial cells (HUAEC) more than 30× more efficiently than either compound separately [Wang 2004]. This finding pointed out the potential importance of the hydrophobic portions of cationic lipids: it appeared that the non-polar parts may be important for the effectiveness of these transfection agents. Further on, it was found that synergistic enhancement of the lipofection activity could be observed in a variety of other cationic phospholipid mixtures; this increased delivery activity was correlated to mesomorphic phase changes that result from exposure to cellular conditions [L. Wang, R. Koynova, H. Parikh, R. C. MacDonald, Transfection Activity of Binary Mixtures of Cationic O-Substituted Phosphatidylcholine Derivatives: The Hydrophobic Core Strongly Modulates Their Physical Properties and DNA Delivery Efficacy, Biophys. J. 91 (2006) 3692-706; R. Koynova, L. Wang, Y. Tarahovsky, R. C. MacDonald, Lipid Phase Control of DNA Delivery, Bioconjug. Chem. 16 (2005) 1335-1339].

A set of cationic lipids, including compounds frequently used in research and clinic such as dimethylammonium-bromides and trimethylammonium-propane, were found to exhibit synergy in binary mixtures. In an attempt to rationalize this finding, performed structural and thermodynamic analysis of those mixtures were performed. Besides the mesomorphic liquid crystalline phases of different topology, lipid-water systems form the lamellar gel (solid) phase, and the gel-liquid crystalline (chain melting) phase transition known as the major energetic event (‘main’ transition) in these systems. Since most of the compounds herein mentioned have saturated hydrocarbon chains, they exhibit solid-liquid crystalline phase transition at temperatures relevant to many practical (clinical) applications; the same is true for their mixtures.

As such, one embodiment of the present invention provides compositions and methods useful in clinical applications for inserting nucleic acids or other agents into cells and tissues, for example, for gene therapy of genetic diseases and disorders or for research uses.

Bilayer properties such as permeability, compressibility, fusogenicity, structural fluctuations, leakiness, etc., exhibit maximum incidental to the chain-melting transition [Mouritsen, O. G., Jorgensen, K., and Honger, T. (1995) Permeability of Lipid Bilayers Near the Phase Transition (E. A. Disalvo and S. A. Simon), pp. 137-160, Boca Raton. 4-10; J. Vanderbosch, H. M. Mcconnell, Fusion of Dipalmitoylphosphatidylcholine Vesicle Membranes Induced by Concanavalin-A, Proc. Natl. Acad. Sci. U.S.A. 72 (1975) 4409-4413; J. H. Prestegard, B. Fellmeth, Fusion of Dimyristoyllecithin Vesicles As Studied by Proton Magnetic-Resonance Spectroscopy, Biochemistry 13 (1974) 1122-1126; F. J. Martin, R. C. MacDonald, Phospholipid Exchange Between Bilayer Membrane-Vesicles, Biochemistry 15 (1976) 321-327; D. Papahadjopoulos, K. Jacobson, S. Nir, T. Isac, Phase-Transitions in Phospholipid Vesicles—Fluorescence Polarization and Permeability Measurements Concerning Effect of Temperature and Cholesterol, Biochim. Biophys. Acta 311 (1973) 330-348; M. C. Blok, L. L. M. Vandeenen, J. Degier, Effect of Gel to Liquid-Crystalline Phase-Transition on Osmotic Behavior of Phosphatidylcholine Liposomes, Biochim. Biophys. Acta 433 (1976) 1-12; D. Marsh, A. Watts, P. F. Knowles, Evidence for Phase Boundary Lipid—Permeability of Tempo-Choline Into Dimyristoylphosphatidylcholine Vesicles at Phase-Transition, Biochemistry 15 (1976) 3570-3578].

As such, an explicit correlation between the transfection efficacy of the cationic lipid mixtures (including saturated-chain compounds) and their solid-liquid crystalline phase transition: specifically, compositions, which are within the two-phase transition region at physiological temperature, exhibit maximum transfection activity is herein demonstrated.

A number of physical and physico-chemical factors have been suggested as lipofection modulators, but the specific route of DNA delivery by cationic lipid vectors is still mostly unknown and at present their efficiency of delivery is unsatisfactorily low for many applications.

A recent and particularly effective strategy for enhancing transfection activity involved combining two cationic lipid derivatives having the same head group but different hydrocarbon chains (Wang 2004). Such combinations often synergistically enhance transfection and allow optimizing activity by merely varying the ratio of the two components. An extensive study, including variety of cationic phosphatidylcholine derivatives with unusual hydrophobic moieties, showed that synergistic enhancement of the lipofection activity could be observed in multiple other cationic phospholipid mixtures (Wang 2006).

It is contemplated that various physical phenomena are responsible for the superior transfection activity of cationic lipid mixtures relative to single lipid formulations. A correlation was previously described between the delivery efficiency of the lipid DNA carriers and the mesomorphic phases they form after interaction with negatively charged membrane lipids. For example, mixed formulations that are particularly effective DNA carriers form phases of highest negative interfacial curvature when interacting with negatively charged membrane lipids, whereas less effective formulations form phases of lower curvature under the same conditions (Koynova 2005). The bilayer bending constant is lowered in lipid mixtures as a result of a mixed bilayer assuming different compositions in the two opposing monolayers; the magnitude of the bending constant reduction increases with increasing difference between the two amphiphiles with respect to charge, head group size, and tail length [M. Bergstrom, Thermodynamics of Unilamellar Vesicles: Influence of Mixing on the Curvature Free Energy of a Vesicle Bilayer, J. Colloid Interface Sci. 240 (2001) 294-306]. Thus, mixed bilayers exhibit higher tendency to form curved nonlamellar arrays. Formation of nonlamellar phases as a result of the interaction of the cationic lipid carriers with the negatively charged membrane lipids is suggested to strongly facilitate DNA release.

Another physico-chemical factor is demonstrated herein, apparently relevant to the process of DNA release, which also promotes transfection success. This is the solid-liquid crystalline phase transition, taking place in the cationic lipid carriers. Critical bilayer properties with respect to delivery, such as permeability and contents release, are known to exhibit maximum during the chain-melting transition. Dramatic increase of the isothermal compressibility of the bilayer near the phase transition temperature has been reported as well [W. Schrader, H. Ebel, P. Grabitz, E. Hanke, T. Heimburg, M. Hoeckel, M. Kahle, F. Wente, U. Kaatze, Compressibility of Lipid Mixtures Studied by Calorimetry and Ultrasonic Velocity Measurements, Journal of Physical Chemistry B 106 (2002) 6581-6586].

For single-component lipid bilayers, the solid-liquid crystalline phase transition proceeds as a highly cooperative event, within a very narrow temperature interval (usually <0.1° C.), and is reasonably well approximated as a first-order phase transition. It is thus impractical to attempt to take advantage of the transition-enhanced bilayer permeability using single-component lipid carriers. For lipid mixtures, on the other hand, the solid-liquid crystalline phase transition is considerably broadened in temperature, even for mixtures with close to ideal mixing. Therefore, solid and liquid crystalline domains of different composition coexist in a relatively broad temperature interval, thus producing boundary defects of higher permeability, and markedly increasing the probability for contents release. Moreover, including two or more cationic lipids instead of one increases the degrees of freedom in the lipoplex-membrane interactions, allowing for larger variation of membrane curvature, as discussed above (Bergstrom 2001) which is likely to be important in membrane fusion.

With the five exemplary binary cationic lipid mixtures discussed herein, a clear correlation of the high-efficiency formulations for each mixture with the compositions was found, for which the lipid bilayers are within the phase transition (two-phase) region, thus exhibiting solid-liquid crystalline phase coexistence. These comparison data are now summarized in FIG. 6. The transfection efficacy (TE) of each composition studied was compared to the average efficacy for the given mixture as: (TE−<TE>)/<TE>, in %, and plotted against the difference of the phase line (liquidus or solidus) temperature for the given composition and 37° C. [T(liquidus)−37° C.] or [37° C.−T(solidus)]. There is a correlation between higher-than-average transfection activity and the lipid composition falling within the transition region, below the liquidus line. There is no apparent correlation, however, between how distant the position of the phase line is from 37° C. at given composition, and the transfection efficacy of that composition. Thus, the increased activity is not a simple temperature effect, but clearly a transition (two-phase) region effect.

In order for nucleic acids (e.g., DNA, RNA) to enter the cell nucleus where it is transcribed, it needs to be released from the lipoplexes after its delivery inside the cell. For example, DNA release was suggested as a critical step along the transfection route, with the extent of DNA release closely correlating with the transfection efficacy (Wang 2006). In order for DNA to be released from lipoplexes, the cationic lipid electrostatic charge should be neutralized. According to current understanding, cellular anionic lipids are responsible for the neutralization of cationic lipid. Indeed, addition of negatively charged liposomes to lipoplexes results in dissociation of DNA from the lipid [R. C. MacDonald, G. W. Ashley, M. M. Shida, V. A. Rakhmanova, Y. S. Tarahovsky, D. P. Pantazatos, M. T. Kennedy, E. V. Pozharski, K. A. Baker, R. D. Jones, H. S. Rosenzweig, K. L. Choi, R. Z. Qiu, T. J. McIntosh, Physical and Biological Properties of Cationic Triesters of Phosphatidylcholine, Biophys. J. 77 (1999) 2612-2629; Y. H. Xu, F. C. Szoka, Mechanism of DNA Release From Cationic Liposome/DNA Complexes Used in Cell Transfection, Biochemistry 35 (1996) 5616-5623; O. Zelphati, F. C. Szoka, Mechanism of Oligonucleotide Release From Cationic Liposomes, Proc. Natl. Acad. Sci. U.S.A. 93 (1996) 11493-11498; G. W. Ashley, M. M. Shida, R. Qiu, M. K. Lahiri, P. C. Levisay, R. D. Jones, K. A. Baker, R. C. MacDonald, Phosphatidylcholinium Compounds: A New Class of Cationic Phospholipids With Transfection Activin and Unusual Physical Properties (Abstract), Biophys. J. 70 (1996) 88; Y. S. Tarahovsky, R. Koynova, R. C. MacDonald, DNA Release From Lipoplexes by Anionic Lipids: Correlation With Lipid Mesomorphism, Interfacial Curvature, and Membrane Fusion, Biophys. J. 87 (2004) 1054-1064]. Neutralization of cationic lipid carriers by anionic membrane lipids, required for DNA release, presupposes lipid exchange between cationic lipoplexes and negatively charged membranes of cytoplasm, most likely by fusion of cell membranes with lipoplexes. It was thus proposed that fusion between cationic bilayers and endosome membrane (or other cellular membrane) leads to displacement of DNA from the lipoplexes. Indeed, it was found previously that highly efficient lipoplex formulations such as EDOPC/diC14DAB 20:80 and EDOPC/DMTAP 67:33 (mol/mol) exhibit also higher fusion ability with negatively charged membrane-mimicking liposomes relative to the pure EDOPC lipoplexes. Furthermore, more efficient DNA escape from those highly efficient lipoplexes has been also verified by fluorescent microscopy. With the structural and thermodynamic results reported here, the enhanced fusogenicity and DNA release from the lipoplexes of superior efficacy can be attributed to the solid-liquid crystalline phase transition taking place at physiological temperature.

Thus, in some embodiments, the present invention provides new mixtures [e.g., excluding mixtures described in Wang 2004; Wang 2006 and those as described in J. Vanderbosch, H. M. Mcconnell, Fusion of Dipalmitoylphosphatidylcholine Vesicle Membranes Induced by Concanavalin-A, Proc. Natl. Acad. Sci. U.S.A. 72 (1975) 4409-4413; J. H. Prestegard, B. Fellmeth, Fusion of Dimyristoyllecithin Vesicles As Studied by Proton Magnetic-Resonance Spectroscopy, Biochemistry 13 (1974) 1122-1126; F. J. Martin, R. C. MacDonald, Phospholipid Exchange Between Bilayer Membrane-Vesicles, Biochemistry 15 (1976) 321-327; S. T. Sun, C. C. Hsang, E. P. Day, J. T. Ho, Fusion of Phosphatidylserine and Mixed Phosphatidylserine-Phosphatidylcholine Vesicles—Dependence on Calcium Concentration and Temperature, Biochim. Biophys. Acta 557 (1979) 45-5; S. T. Sun, E. P. Day, J. T. Ho, Temperature-Dependence of Calcium-Induced Fusion of Sonicated Phosphatidylserine Vesicles, Proc. Natl. Acad. Sci. U.S.A. 75 (1978) 4325-4328; F. J. Martin (1975) Model Systems Applied to Study of Membrane-Membrane Interactions, PhD Thesis, Northwestern University] that provide improved transfection properties. Indeed, as previously discussed, solid and liquid crystalline domains of necessarily different compositions coexist within the transition region, which inevitably generates packing defects. Such packing defects facilitate the fusion of bilayers, because bilayer fusion, at some stage in the process, involve merging of the hydrophobic cores of the two participating membranes.

As such, experimentation in developing embodiments of the present invention demonstrates that higher fusogenicity is an attribute of, even though not a guarantee for, superior transfection activity. It is demonstrated herein that manipulating the composition of the lipid carriers so as to adjust their phase transition to take place at physiological temperature provides a valuable approach to enhance their nucleic acid delivery efficacy.

In one embodiment, the present invention provides compositions and methods for transferring nucleic acids or other agents into cells and tissues using a mixture of lipids. In some embodiments, the mixture of lipids resides within the solid-liquid crystalline two-phase region of a binary phase diagram at physiological temperature. In some embodiments, said transfer of nucleic acids is performed in vitro or in vivo. In some embodiments, the mixture of lipid is one or more of EDMPC/EDPPC, diC14DAB/diC18DAB, EDOPC/diC14DAB, EDOPC/diC18DAB, and EDOPC/DMTAP. In some embodiments, choice of lipid length, saturation, concentration, etc. are selected to achieve the desired property. Candidate mixtures may be assessed by the techniques described herein to identify mixtures having the desired phase transition at a desired temperature. In some embodiments, the mixtures exclude lipid combinations known in the art. However, the present invention is not limited to the combination of lipids used, and any combination of lipid that resides within the solid-liquid crystalline two-phase region of a binary phase diagram are contemplated for use in the present invention.

In one embodiment, the nucleic acids associated with a mixture of lipids as described herein for transfecting cells or tissues comprise DNA or RNA. In some embodiments, the nucleic acids are preferably incorporated into an expression vector. In some embodiments, the expression vector is of plasmid or viral origin. In some embodiments, the expression vector comprises DNA encoding a protein of interest (e.g., reporter gene, enzyme, transcription factor, functional cellular protein, metabolic cellular protein, etc.) or fragment thereof (e.g., encoding a functional or non-functional protein) operably linked to a promoter, wherein said promoter is cell or tissue specific (e.g., only transcribes genetic information in a particular cell or tissue type) or is not tissue specific (e.g., transcribes genetic information in cells regardless of origin). In some embodiments, in vivo transfection comprises gene therapy for treating diseases and disorders. In some embodiments, said gene therapy treatment using the compositions and methods of the present invention are used in conjunction with other established therapies.

In one embodiment, a mixture of lipids as described herein is complexed with small interfering RNAs (siRNA) or other constructs that produce RNAi sequences such that transfection of such constructs using the compositions and methods of the present invention into cells causes one or more gene products not to be produced by the cell.

### EXPERIMENTAL

The following examples are provided in order to demonstrate and further illustrate certain preferred embodiments and aspects of the present invention and are not to be construed as limiting the scope thereof.

### Example 1

### Lipid Preparation

Ethyl-phosphatidylcholines (1,2-diacyl-sn-glycero-3-ethylphosphocholines) with oleoyl, myristoyl, and palmitoyl chains—EDOPC, EDMPC, EDPPC—was synthesized as previously described [R. C. MacDonald, V. A. Rakhmanova, K. L. Choi, H. S. Rosenzweig, M. K. Lahiri, O-Ethylphosphatidylcholine: A Metabolizable Cationic Phospholipid Which Is a Serum-Compatible DNA Transfection Agent, Journal of Pharmaceutical Sciences 88 (1999) 896-904]. Ditetradecyldimethylammonium-bromide (diC₁₄-DAB) (Aldrich), dioctadecyldimethylammonium-bromide (DiC₁₈-DAB) (Sigma), dimyristoyltrimethylammonium-propane (DMTAP) (Avanti) were used without further purification. The phospholipids migrated as a single spot by thin-layer chromatography.

### Example 2

### Sample Preparation

Chloroform solutions of cationic lipids were mixed in glass vials at the desired ratios, solvent was removed with a N₂ stream and, subsequently, high vacuum. The lipid mixtures were hydrated in PBS (50 mM phosphate buffer, 100 mM NaCl, pH 7.2) overnight at room temperature and vortex-mixed for several minutes; then they were subjected to several cycles of freezing and thawing. Samples were subsequently equilibrated overnight at room temperature before measurement.

### Example 3

### DSC Measurements

DSC measurements were performed using a VP-DSC Microcalorimeter (MicroCal Inc., Northampton, Mass.) at 30° C./h. Thermograms were analyzed using OriginLab (Northampton, Mass.) software. The onset and the completion temperatures of the phase transitions needed for the construction of the phase diagram were determined by the intersections of the peak slopes with the baseline on the thermograms. The maximums of the heat capacity curves were taken as the phase transition temperatures of the pure components. For asymmetric or split line shapes of the composite aggregates, the transition temperature was defined as that temperature at which the area of the endotherm was divided into equal halves.

### Example 4

### Synchrotron Small-Angle X-Ray Diffraction (SAXD)

Measurements were performed at Argonne National Laboratory, Advanced Photon Source, DND-CAT (beamline 5-IDD) and BioCAT (beamline 18-ID), using 12 keV X-rays, as previously described [1 R. Koynova, R. C. MacDonald, Mixtures of Cationic Lipid O-Ethylphosphatidylcholine With Membrane Lipids and DNA: Phase Diagrams, Biophys. J. 85 (2003) 2449-2465]. The lipid concentration of the dispersions was 20 wt %. Samples were filled into glass capillaries (Charles Supper Co., Natick, Mass.) and flame-sealed. A Linkam thermal stage (Linkam Sci Instruments, Surrey, England) provided temperature control. Linear heating and cooling scans were performed at rates of 1-5° C./min. Exposure times were typically 0.5 sec. Data were collected using a MAR-CCD detector. Sample-to-detector distance was 1.8-2 m. Diffraction intensity vs. Q plots were obtained by radial integration of the 2D patterns using the interactive data-evaluating program FIT2D [1 A. P. Hammersley, S. O. Svensson, M. Hanfland, A. N. Fitch, D. Hausermann, Two-Dimensional Detector Software: From Real Detector to Idealised Image or Two-Theta Scan, High Pressure Research 14 (1996) 235-248]. Some samples with longer exposure time were checked by thin layer chromatography after the experiments. Products of lipid degradation were not detected in these samples, and radiation damage of the lipids was not evident from their X-ray patterns.

### Example 5

### Cell Culture and Transfection Protocols

Human umbilical artery endothelial cells (HUAECs) were obtained from Cambrex (Walkersville, Md.) were and maintained in EGM-2 MV containing 5% fetal bovine serum (FBS) (Cambrex) at 37° C. with 5% CO₂. At confluence, the cells were passaged using 0.25 mg/ml Trypsin/EDTA (Cambrex) and were used at passages 5-10 for these experiments. It is noted that human endothelial cells, and particularly HUAECs, are known in the art to be difficult to transfect (see, Teifel, et al., Endothelium, New York, 5, 21-35 (1997); Krotz et al., J. Vasc. Res., 40:425-434 (2003); and Tanner et al., Cardiovascular Research, 35:522-528 (1997)) all of which are herein incorporated by reference. For example, it has been reported that the level of transfection for a reporter gene in human umbilical vein endothelial cells is less than 1% of that of HeLa cells (Edgell et al., BioTechniques, 25:264-272 (1998), herein incorporated by reference).

The cells were seeded in 96-well plates at 24 h before transfection at densities appropriate to give about 80% confluence at the time of transfection. CMV-β-galactosidase plasmid was purchased from Clontech Laboratories Inc. (Palo Alto, Calif.) and propagated and purified by Bayou Biolabs (Harahan, La.). Cationic lipid mixtures were hydrated in HBSS at temperature higher than the phase transition of the higher-melting component and diluted in Opti-MEM, then pipetted at that temperature into an equal volume of plasmid DNA solution (also diluted in Opti-MEM) as previously described [1 L. Wang, R. C. MacDonald, New Strategy for Transfection: Mixtures of Medium-Chain and Long-Chain Cationic Lipids Synergistically Enhance Transfection, Gene Ther. 11 (2004) 1358-1362]. The resultant DNA-lipid complexes were equilibrated for 15 min and then added to cells. At 2 h after their addition, the lipoplexes were removed and fresh medium was added. Cells were assayed for β-galactosidase activity 24 h after transfection with a microplate fluorometric assay [1 V. A. Rakhmanova, R. C. MacDonald, A Microplate Fluorimetric Assay for Transfection of the Beta-Galactosidase Reporter Gene, Analytical Biochemistry 257 (1998) 234-237].

### Results

### EDMPC/EDPPC

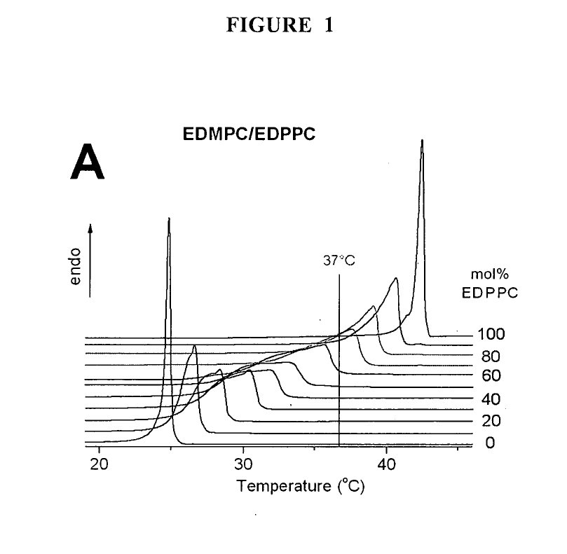

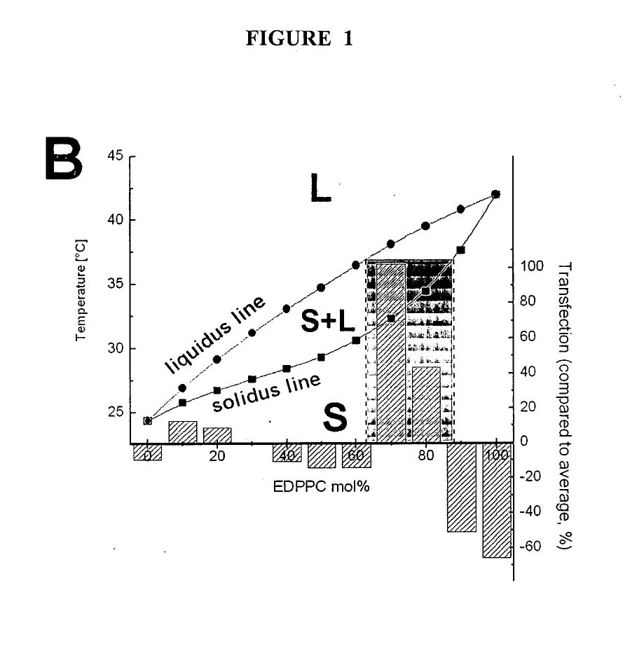

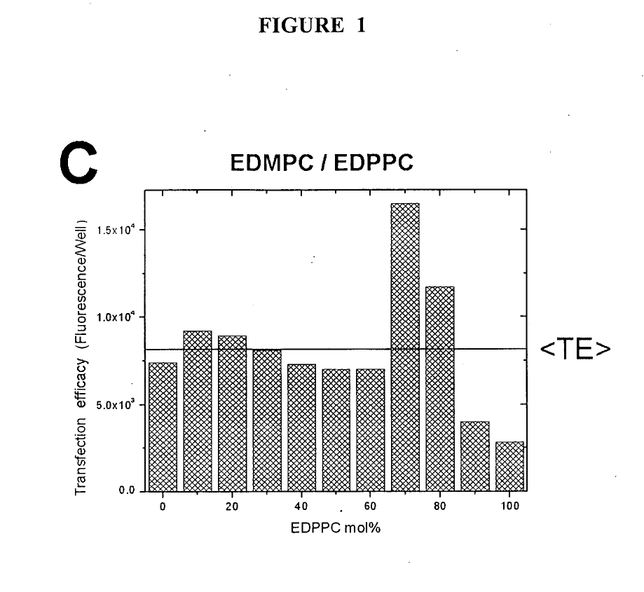

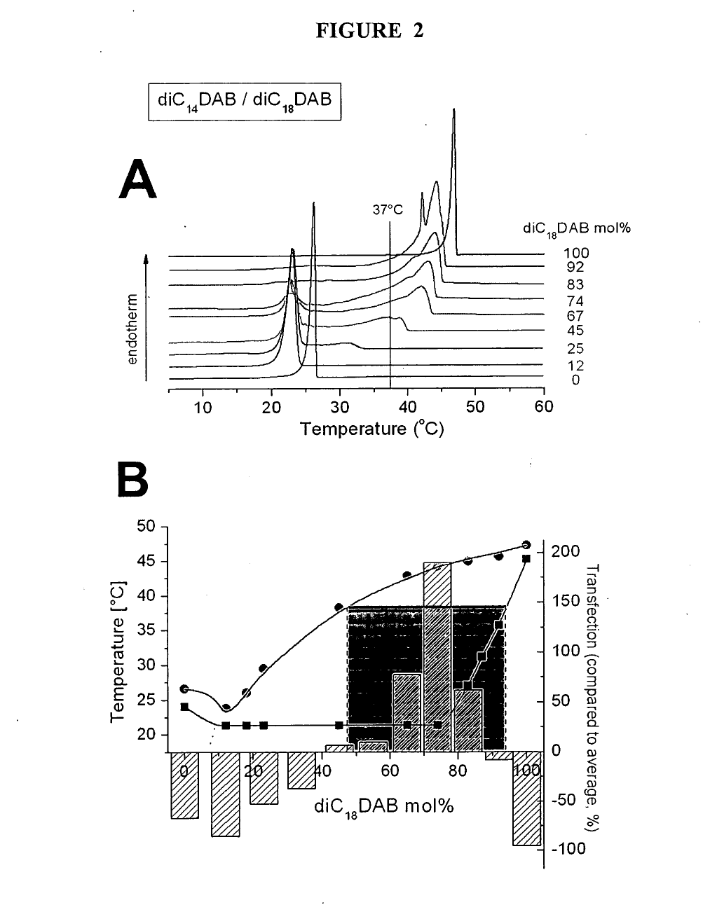

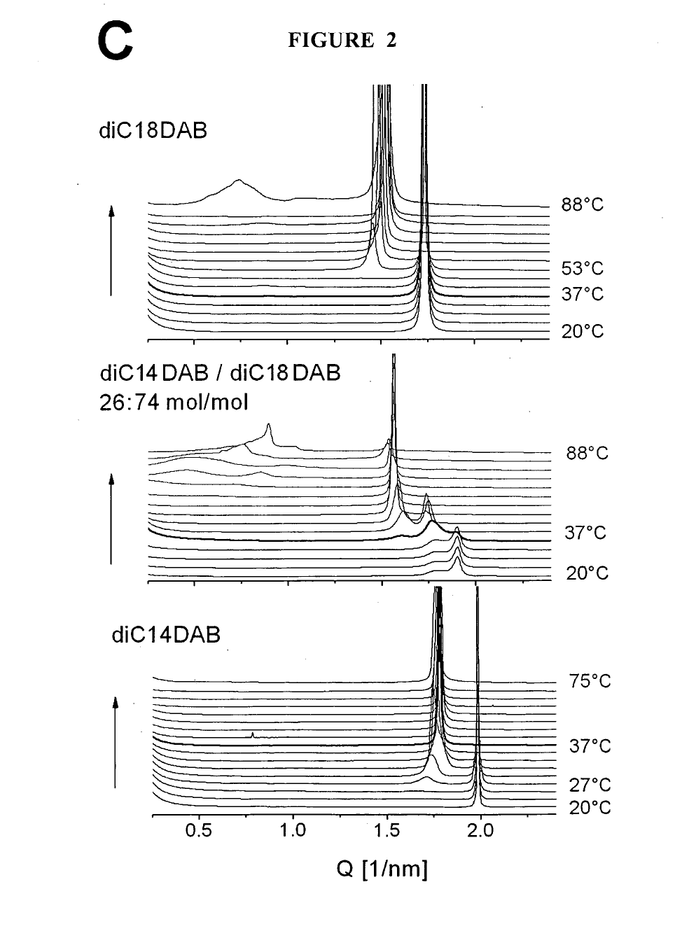

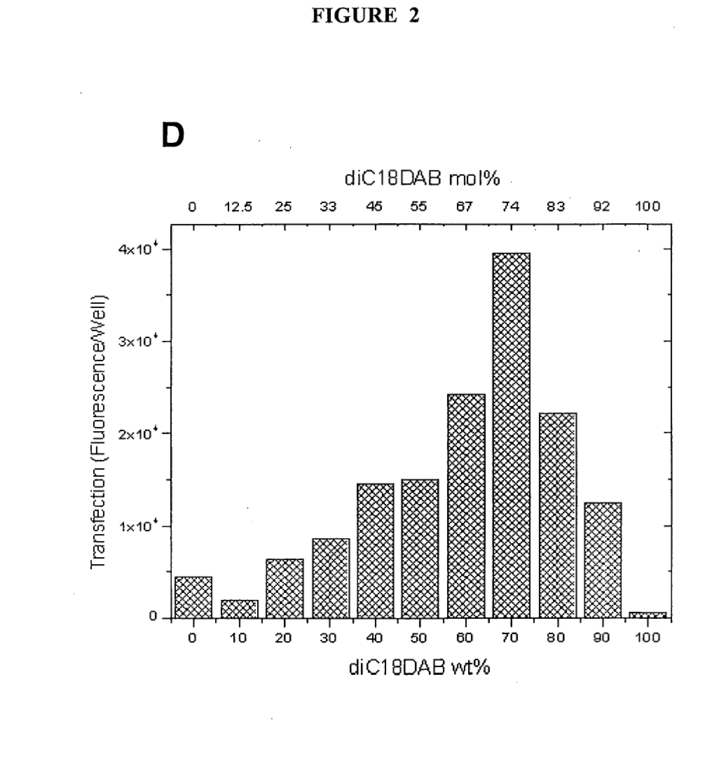

A selection of calorimetric thermograms of aqueous dispersions of mixtures of EDMPC and EDPPC at different mole ratios is shown in FIG. 1A. Pure hydrated EDMPC exhibited a melting transition at Tₘ=24° C., EDPPC—at 42° C., as reported earlier [R. C. MacDonald, G. W. Ashley, M. M. Shida, V. A. Rakhmanova, Y. S. Tarahovsky, D. P. Pantazatos, M. T. Kennedy, E. V. Pozharski, K. A. Baker, R. D. Jones, H. S. Rosenzweig, K. L. Choi, R. Z. Qiu, T. J. McIntosh, Physical and Biological Properties of Cationic Triesters of Phosphatidylcholine, Biophys. J. 77 (1999) 2612-2629; R. Koynova, R. C. MacDonald, Columnar DNA Superlattices in Lamellar O-Ethylphosphatidylcholine Lipoplexes: Mechanism of the Gel-Liquid Crystalline Lipid Phase Transition, Nano Letters 4 (2004) 1475-1479; R. Koynova, R. C. MacDonald, Lipid Transfer Between Cationic Vesicles and Lipid-DNA Lipoplexes. Effect of Serum, Biochim. Biophys. Acta-Biomembranes 1714 (2005) 63-70]. The phase diagram (FIG. 1B) of this mixture was constructed based on the DSC data [1 R. Koynova, R. C. MacDonald, Lipid Transfer Between Cationic Vesicles and Lipid-DNA Lipoplexes. Effect of Serum, Biochim. Biophys. Acta-Biomembranes 1714 (2005) 63-70]. According to it, EDMPC/EDPPC mixtures with up to 60 mol % EDPPC are in the liquid crystalline (L) phase at physiological temperature; mixtures with >90 mol % EDPPC are in the solid (S) phase. Mixtures with 70-80 mol % EDPPC reside within the phase transition two-phase region, between the solidus and liquidus lines of the phase diagram, and are thus expected to exhibit solid-liquid crystalline (S+L) phase coexistence at 37° C. (FIG. 2A).

The transfection activity of EDMPC/EDPPC mixed lipoplexes was tested with HUAEC cells in vitro. EDMPC lipoplexes are better transfection agents than EDPPC; this is expected since the former lipid is in the liquid crystalline phase, while the latter is in the solid phase at physiological temperatures (FIG. 1C). It is noteworthy that the solid-phase EDPPC still transfects DNA into cells, provided the lipoplexes are formed at T>Tₘ. The mixed EDMPC/EDPPC lipoplexes exhibit a pronounced maximum in their transfection activity at 70-80 mol % EDPPC; the compositions for which solid-liquid crystalline phase coexistence is characteristic at 37° C. The transfection efficacy of each sample was compared to the average efficacy typical for this mixture (the red line in FIG. 1C) calculated from all the formulations that were examined (FIG. 1B, columns). Particularly higher-than-average activity is obvious for the 70-80 mol % EDPPC mixtures, which exhibited solid-liquid crystalline phase coexistence at 37° C.

diC14DAB/diC18DAB

A selection of calorimetric thermograms of aqueous dispersions of mixtures of diC14DAB and diC18DAB at different mole ratios is shown in FIG. 2A. Pure hydrated diC14DAB exhibited a melting transition at Tₘ=26° C., diC18DAB—at ˜50° C., as reported [1 P. C. Schulz, J. E. Puig, G. Barreiro, L. A. Torres, Thermal Transitions in Surfactant-Based Lyotropic Liquid-Crystals, Thermochim. Acta 231 (1994) 239-256]. The melting transition of diC14DAB/diC18DAB mixtures at 10-75 mol % diC18DAB starts at ˜20° C., i.e., at lower temperature than the pure compounds. The phase diagram constructed on the basis of the calorimetric results is depicted in FIG. 2B. It is a eutectic phase diagram, with a eutectic point at ˜10% diC18DAB. The horizontal portion of the solidus line indicates a phase separation in the solid phase for samples of 10-75 wt % diC18DAB—i.e., two solid phases of different composition coexist in these mixtures at T<20° C. Upon heating, the samples enter an extended solid-liquid crystalline phase coexisting region; for diC18DAB content between ˜45 mol % and 90 mol %, it extends to above physiological temperature.

Thus, according to the temperature-composition phase diagram (FIG. 2B), at physiological temperature (37° C.), diC14DAB/diC18DAB mixtures with ≦45 mol % diC18DAB are in the liquid crystalline phase; mixtures with >90 mol % diC18DAB are in the solid (gel) phase. Mixtures with 45-90 mol % diC18DAB reside within the solid-liquid crystalline phase coexistence region, between the solidus and liquidus lines of the phase diagram (FIG. 2B). Thus, solid and liquid crystalline domains are expected to coexist in the lipid bilayers of these samples at 37° C.

Small-angle X-ray diffraction (SAXD) measurements were performed on diC14DAB, diC18DAB, and diC14DAB/diC18DAB 26:74 mol/mol mixture, the composition of maximum transfection activity (FIG. 2D). The diffraction profiles recorded upon heating scans 20-90° C. are presented in FIG. 2C. At 20° C., diC18DAB forms a lamellar solid phase with rather short lamellar repeat distance, d=3.63 nm; it has been previously ascribed to molecular tilt with respect to the bilayer surface [M. Jung, A. L. German, H. R. Fischer, Polymerisation in Lyotropic Liquid-Crystalline Phases of Dioctadecyldimethylammonium Bromide, Colloid Polym. Sci. 279 (2001) 105-113; K. Okuyama, Y. Soboi, K. Hirabayashi, A. Harada, A. Kumano, T. Kaziyama, M. Takayanagi, T. Kunitake, Single-Crystals of Totally Synthetic Amphiphiles, Dialkyldimethylammonium Bromides, Chem. Lett. (1984) 2117-2120]. Upon heating, at 47° C. it undergoes a transition to another lamellar phase with a larger lamellar period, d=4.2 nm. According to the DSC results (transition enthalpy), this corresponds to the solid-liquid crystalline transition. The shorter chain compound, diC14DAB, also arrange into lamellar phase with a short lamellar repeat period, 3.17 nm, at 20° C. At 25° C., it undergoes a transition to another lamellar (liquid crystalline) phase with d=4.53 nm. The diC14DAB/diC18DAB 26:74 mol/mol mixture exhibits an indication of phase coexistence at low temperature, as expected from the binary phase diagram: two diffraction peaks are observed at 3.3 nm and 3.5 nm. Structural rearrangement takes place at about physiological temperature, between 35-45° C., to a homogeneous lamellar (liquid crystalline) phase with d=4.02 nm. At still higher temperatures, ˜67-70° C., additional reflexes appear at rather small angles; at ˜85° C. the major peaks spacings are 7.04 nm and 6.10 nm, in the ratio 1/√6:1/√8, characteristic of the Ia3d cubic phase.

Maximum transfection activity for this cationic lipid mixture was at diC14DAB/diC18DAB 26:74 mol/mol (FIG. 2D). The transfection efficacy of each sample was compared to the average efficacy typical for this mixture calculated from all the formulations that were examined (FIG. 2B, columns). The distinctly higher-than-average activity is demonstrated by the 60-90 mol % diC18DAB mixtures, for which solid-liquid crystalline phase coexistence is observed at 37° C.

### EDOPC/diC14DAB

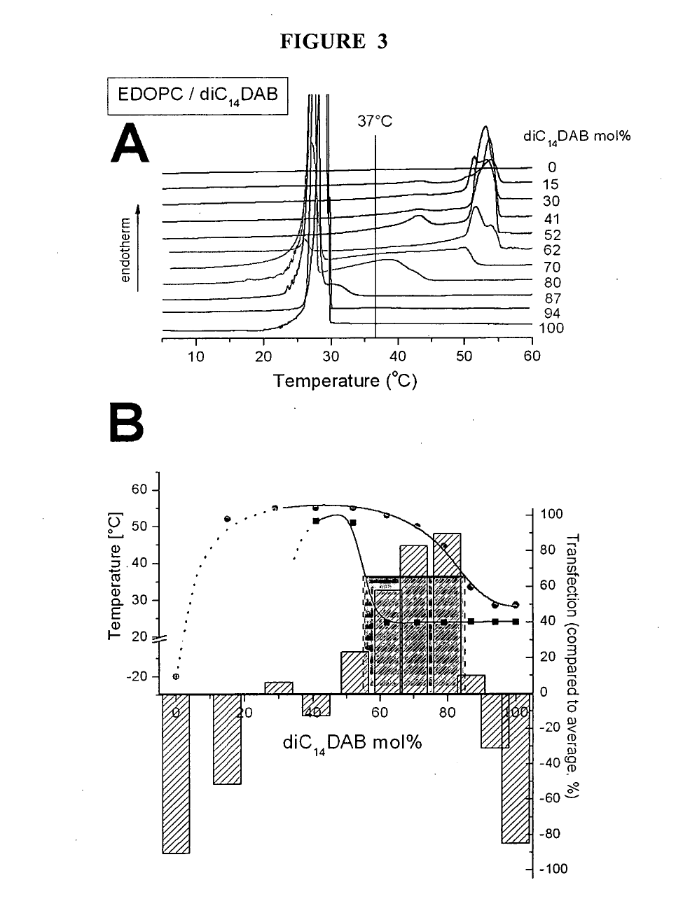

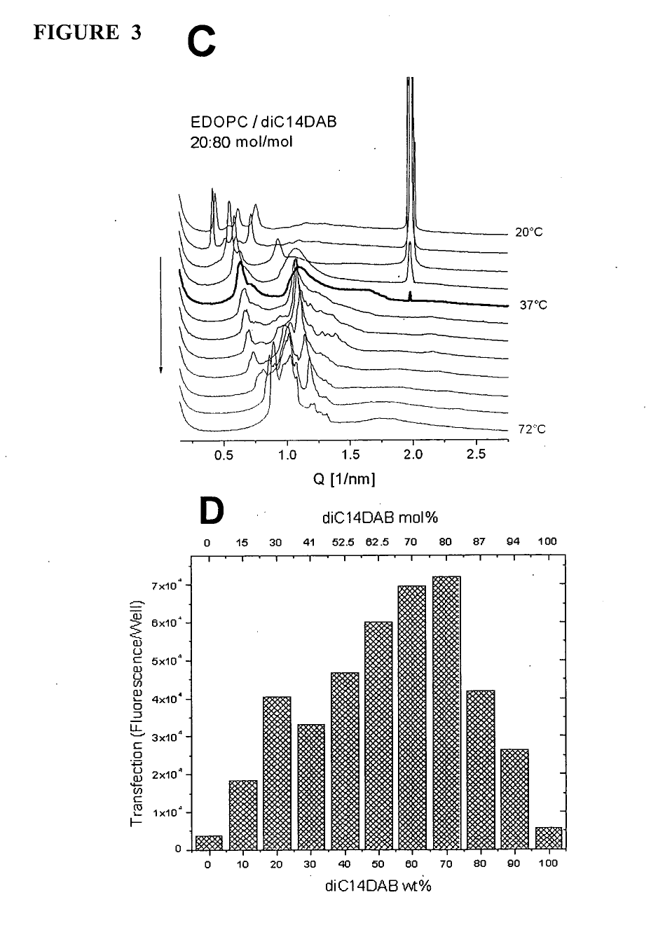

A selection of thermograms of aqueous dispersions of mixtures of EDOPC and diC14DAB at different mole ratios is shown in FIG. 3A. No thermal events were recorded at the thermogram of EDOPC in the range 0-100° C. According to previous experience, cationic ethyl derivatives of the phosphatidylcholines exhibit solid-liquid crystalline transitions at temperatures coinciding with those for the parent phosphatidylcholines [R. C. MacDonald, G. W. Ashley, M. M. Shida, V. A. Rakhmanova, Y. S. Tarahovsky, D. P. Pantazatos, M. T. Kennedy, E. V. Pozharski, K. A. Baker, R. D. Jones, H. S. Rosenzweig, K. L. Choi, R. Z. Qiu, T. J. McIntosh, Physical and Biological Properties of Cationic Triesters of Phosphatidylcholine, Biophys. J. 77 (1999) 2612-2629; R. Koynova, R. C. MacDonald, Columnar DNA Superlattices in Lamellar O-Ethylphosphatidylcholine Lipoplexes Mechanism of the Gel-Liquid Crystalline Lipid Phase Transition, Nano Letters 4 (2004) 1475-147; R. Koynova, R. C. MacDonald, Cationic O-Ethylphosphatidylcholines and Their Lipoplexes: Phase Behavior Aspects, Structural Organization and Morphology, Biochim. Biophys. Acta-Biomembranes 1613 (2003) 39-48]. Thus, EDOPC is expected to have a Tm˜−20° C., similarly to DOPC [Lipid Data Bank, http://www.ldb.chemistry.ohio-state.edu/.2000]. Although EDOPC is supposedly characterized by a low transition temperature, addition of EDOPC to diC14DAB results in the appearance of a high-temperature component of the diC14DAB transition endotherm. At ˜40 mol % diC14DAB, single endotherm is observed at 53° C., with enthalpy comparable to that of the melting transition of pure diC14DAB. At lower diC14DAB content (15-25 mol %), only low-enthalpy endotherms are recorded at high temperatures in the 0-100° C. range, while at <15 mol % there were no observable thermal events in the thermograms.

Based on the DSC data, a partial temperature-composition phase diagram of the mixture was constructed (FIG. 3B). Since the phase transition of the pure EDOPC and those of the samples with dominating EDOPC (especially the temperatures of the transition onset) are outside the temperature range accessible by high-sensitivity DSC, the phase lines are tentatively extrapolated for those compositions. The phase diagram is of a high-temperature aseotropic (isoconcentration point) type. Azeotropic phase diagrams have been previously reported for other lipid mixtures containing charged lipids [J. R. Silvius, Anomalous Mixing of Zwitterionic and Anionic Phospholipids With Double-Chain Cationic Amphiphiles in Lipid Bilayers, Biochim. Biophys. Acta 1070 (1991) 51-59; R. Zantl, L. Baicu, F. Artzner, I. Sprenger, G. Rapp, J. O. Radler, Thermotropic Phase Behavior of Cationic Lipid-DNA Complexes Compared to Binary Lipid Mixtures, Journal of Physical Chemistry B 103 (1999) 10300-10310; F. M. Linseisen, S. Bayerl, T. M. Bayerl, H-2-NMR and DSC Study of DPPC-DODAB Mixtures, Chem. Phys. Lipids 83 (1996) 9-23; P. Garidel, C. Johann, A. Blume, Nonideal Mixing and Phase Separation in Phosphatidylcholine Phosphatidic Acid Mixtures As a Function of Acyl Chain Length and PH, Biophys. J. 72 (1997) 2196-2210]. As herein reported, these are usually of the upper isoconcentration point type, in which the transition temperatures of the mixtures at particular compositions are higher than those of the pure components and exhibit a maximum. Analysis of the phase diagrams show that the presence of upper isoconcentration point typically reflect higher values of the excess free energy of mixing (“nonideal” energy) in the liquid than in the solid state at those particular compositions [P. Gordon (1968) Principles of Phase Diagrams in Material Systems, McGraw-Hill, New York]. Such a combination of nonideal energies is conceivable in mixtures with negative nonideal energy in the solid state, in which contacts between unlike molecules are preferred to those between like ones, i.e., when the nearest-neighbor pairs tend to be made up of unlike molecules. Minimization of the electrostatic repulsion in some mixtures containing charged components results in such a tendency, even if the two components are of the same charge. The different structure of the headgroups, with different locations of the charged functional moieties with respect to the bilayer surface accounts for such preferences to a chessboard-like arrangement in the solid phase, minimizing the electrostatic repulsion. The horizontal portion of the solidus line at compositions with dominating diC14DAB indicates phase separation in the solid phase at those compositions below 20° C.

Small-angle X-ray diffraction (SAXD) measurements were performed with samples containing EDOPC and diC14DAB. The structural data for the pure diC14DAB were presented above—it undergoes a solid-liquid crystalline phase transition at ˜26° C. Pure EDOPC has been previously reported to arrange into lamellar phase with lamellar spacing of ˜4.8 nm [R. Koynova, R. C. MacDonald, Cationic O-Ethylphosphatidylcholines and Their Lipoplexes: Phase Behavior Aspects, Structural Organization and Morphology, Biochim. Biophys. Acta-Biomembranes 1613 (2003) 39-48]. The composition of maximum transfection activity, EDOPC/diC14DAB 20:80 mol/mol (FIG. 3D) is characterized by a phase coexistence of a lamellar phase (d=3.17 nm) and a cubic mesomorphic phase of the Im3m topology (indexed by 8 observable diffraction peaks) at 20° C. (FIG. 3C). As expected from the phase diagram, one of the coexisting phases is composed by virtually pure diC14DAB as judged by its lamellar spacing, equivalent to that of the solid-phase diC14DAB. The solid-liquid crystalline phase coexistence is observed up to ˜40° C., i.e., at physiological temperature the sample is characterized by extensive solid-liquid crystalline phase coexistence.

Comparison of the transfection efficacy of samples of different composition to the average efficacy typical for this mixture is included in FIG. 3B (columns). The higher-than-average activity of the formulations of 60-80 mol % diC14DAB coincides with the region of the phase diagram, for which solid-liquid crystalline phase coexistence is characteristic at 37° C.

### EDOPC/diC18DAB

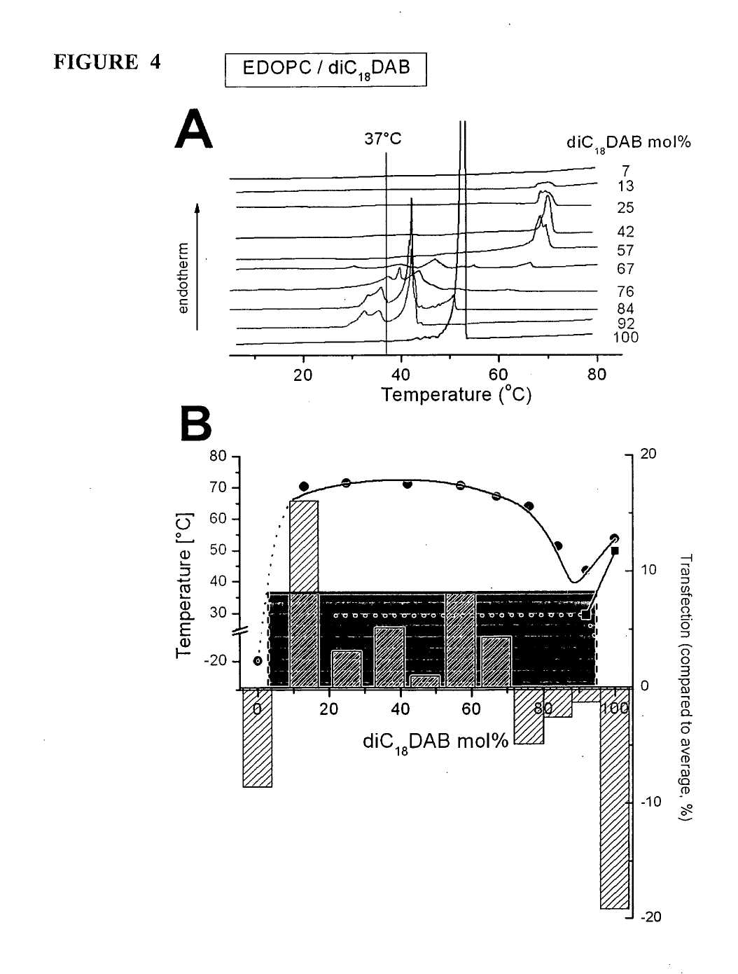

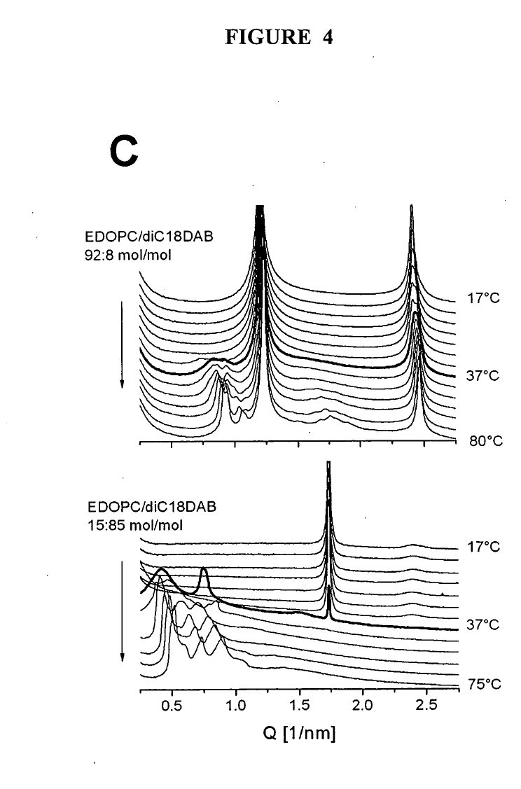

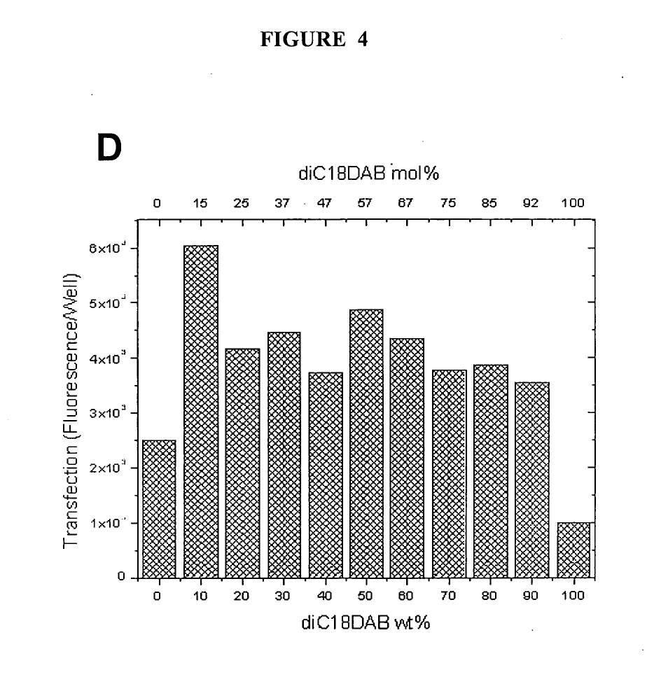

DSC thermograms of samples of different EDOPC/diC18DAB ratios are shown on FIG. 4A. The phase diagram constructed from these data is shown in FIG. 4B. Pure hydrated diC18DAB exhibited a melting transition at ˜50° C. The low enthalpy of the thermal events observed at <90 mol % diC18DAB are presumably an indication that considerable part of the melting (solid-liquid crystalline) transition is initiated at subzero temperatures, where the EDOPC phase transition takes place. A liquidus line of the phase diagram was constructed, delimiting from above the region of solid-liquid crystalline phase coexistence. Since that liquidus line lies generally at >37° C. (except for samples of very high, ≧90 mol %, EDOPC content), the EDOPC/diC18DAB mixtures are expected to exhibit solid-liquid crystalline phase coexistence at physiological temperature for a wide range of compositions. Since pure diC18DAB is in the solid phase at 37° C., it is also outside the coexistence region. The same is presumably true for mixed samples of very high content of that lipid (≧95%).

The data from SAXD experiments with samples of compositions close to the ends of the expected phase coexistence region in the EDOPC/diC18DAB phase diagram, namely with 15 mol % and 92 mol % diC18DAB, corresponded to the calorimetric data (FIG. 4C). Both samples show coexistence of a lamellar and a cubic phase at 37° C.

According to the shape of the phase diagram, solid-liquid crystalline phase coexistence is expected at physiological temperature in a wide composition range, at almost any composition except for the pure compounds. The transfection efficacy of the mixed lipoplexes is thus above or close to the average for this mixture, and better than that of the pure compounds (FIG. 4B, right Y-axis).

### EDOPC/DMTAP

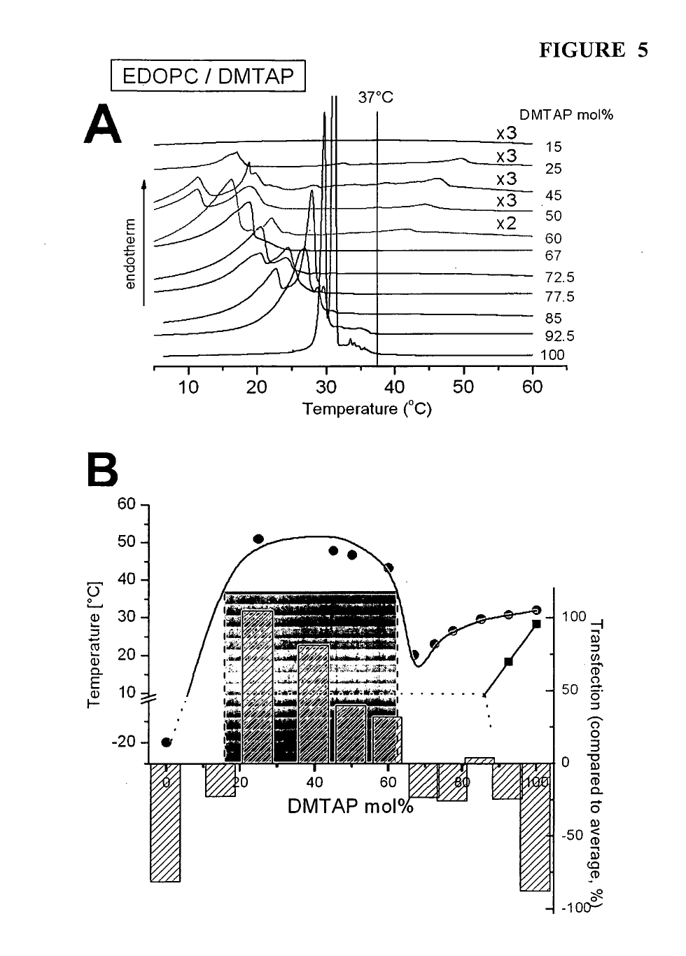

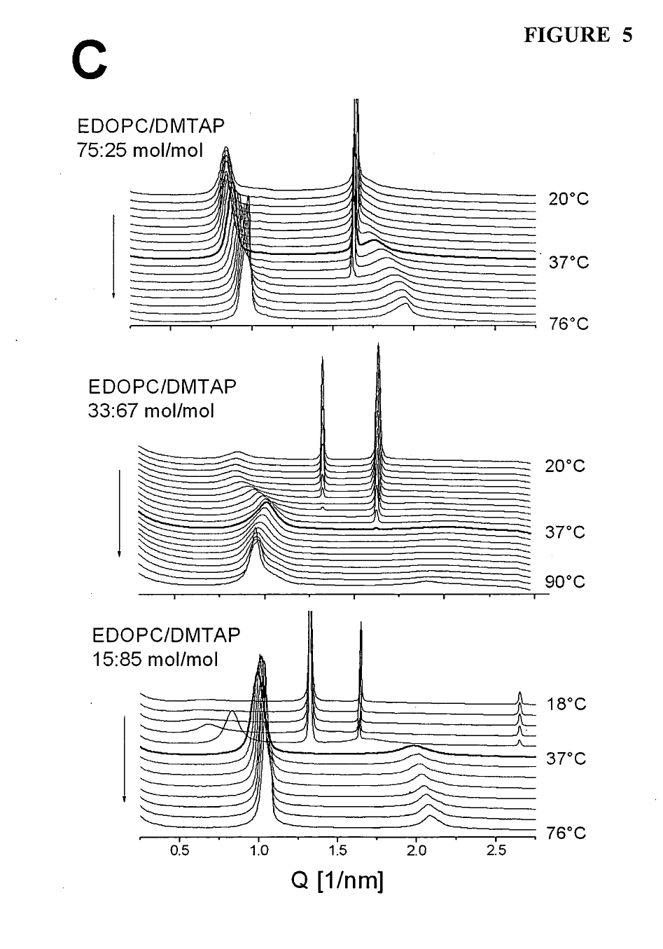

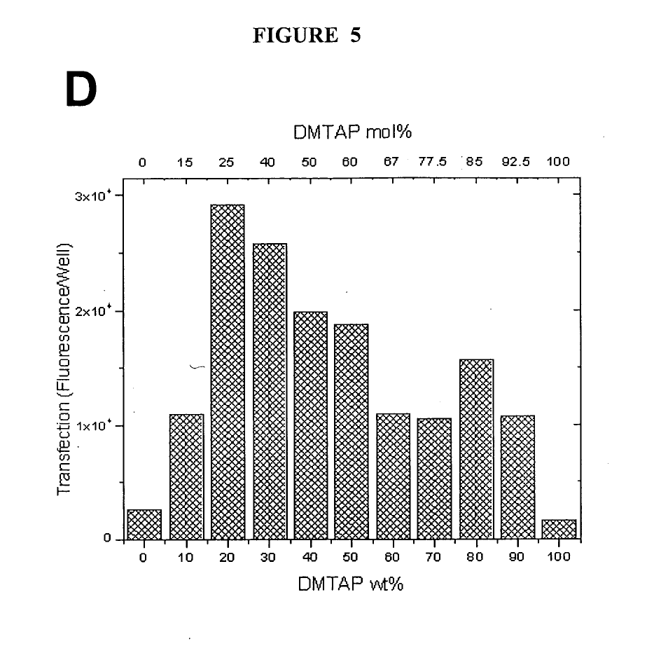

DSC thermograms of samples of different EDOPC/DMTAP ratios are shown in FIG. 5A, and the respective phase diagram constructed based on these data in FIG. 5B. Because of the low Tₘ of EDOPC, the position of the solidus line is only tentatively drafted, especially at dominating EDOPC content (as previously discussed). The phase diagram presumably contains a monotectic point at ˜67 mol % DMTAP and 20° C.

SAXD profiles recorded on heating with samples of three different compositions, EDOPC/DMTAP 75:25, 33:67, and 15:85 (mol/mol), are shown in FIG. 5C. All three compositions exhibit phase separation at 20° C. The samples with dominating DMTAP content, however, become homogeneous liquid crystalline at temperature <37° C. The EDOPC/DMTAP 75:25 mol/mol formulation, the composition of maximum transfection activity (FIG. 5D), is within the transition region at 37° C. and becomes homogeneous only >50° C., in agreement with the phase diagram constructed from the DSC data.

Comparison of the transfection efficacy of samples of different composition to the average efficacy typical for this mixture (FIG. 5B) indicates that the higher-than-average activity of the samples of 20-60 mol % DMTAP mixtures in this case also corresponds to the region in the phase diagram for which solid-liquid crystalline phase coexistence is established at 37° C.

All publications and patents mentioned in the present application are herein incorporated by reference. Various modification and variation of the described methods and compositions of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

We claim:

1. A composition for use as a delivery system comprising a mixture of lipids wherein the mixture resides within the solid-liquid crystalline two-phase region of a binary phase diagram at physiological temperature.

2. The composition of claim 1, wherein said mixture of lipids is selected from the group consisting of:
i) EDMPC and EDPPC,
ii) diC14DAB and diC18DAB,
iii) EDOPC and diC14DAB,
iv) EDOPC and diC18DAB, and
v) EDOPC and DMTAP.

3. The composition of claim 1, wherein said mixture of lipids is selected from the group consisting of:
i) EDMPC and EDPPC, wherein 70-80 mol % of said mixture of lipids is EDPPC;
ii) diC14DAB and diC18DAB, wherein 50-90 mol % of said mixture of lipids is diC18DAB;
iii) EDOPC and diC14DAB, wherein 60-80 mol % of said mixture of lipids is diC14DAB;
iv) EDOPC and diC18DAB, wherein 10-90 mol % of said mixture of lipids is diC18DAB; and
v) EDOPC and DMTAP, wherein 20-60 mol % of said mixture of lipids is DMTAP.

4. The composition of claim 1, further comprising a nucleic acid molecule.

5. The composition of claim 1, further comprising an expression vector.

6. The composition of claim 1, further comprising a drug.

7. A method of transfecting a cell comprising, exposing a cell to a composition comprising:
a) a mixture of lipids wherein the mixture resides within the solid-liquid crystalline two-phase region of a binary phase diagram at physiological temperature; and
b) a nucleic acid molecule.

8. The method of claim 1, wherein said mixture of lipids is selected from the group consisting of:
i) EDMPC and EDPPC,
ii) diC14DAB and diC18DAB,
iii) EDOPC and diC14DAB,
iv) EDOPC and diC18DAB, and
v) EDOPC and DMTAP.

9. The method of claim 1, wherein said mixture of lipids is selected from the group consisting of:
i) EDMPC and EDPPC, wherein 70-80 mol % of said mixture of lipids is EDPPC;
ii) diC14DAB and diC18DAB, wherein 50-90 mol % of said mixture of lipids is diC18DAB;
iii) EDOPC and diC14DAB, wherein 60-80 mol % of said mixture of lipids is diC14DAB;
iv) EDOPC and diC18DAB, wherein 10-90 mol % of said mixture of lipids is diC18DAB; and
v) EDOPC and DMTAP, wherein 20-60 mol % of said mixture of lipids is DMTAP.

10. The method of claim 7, wherein said cell is in vitro.

11. The method of claim 7, wherein said cell is in vivo.

12. The method of claim 7, further comprising an expression vector.

13. The method of claim 7, further comprising a drug.

14. A method for selecting a lipid mixture that provides high transfection efficiency in a delivery system, comprising:
a) selecting a first lipid and a second lipid; and
b) combining a ratio of said first lipid and second lipid in a transfection reagent, said ratio optimized to cause said first and second lipids to exist within a solid-liquid crystalline two-phase region of a binary phase diagram at physiological temperature.

15. A composition comprising a delivery system selected by the method of claim 14.
